(19) United States
(12) Patent Application Publication
(10) Pub. No.: US 2025/0329440 A1
(43) Pub. Date: Oct. 23, 2025

(54) MEDICAL DEVICE, MEDICAL SYSTEM, AND METHOD FOR CONTROLLING A MEDICAL DEVICE AND METHOD FOR DETERMINING THE NEED TO TRAIN A USER OF MEDICAL DEVICE

(71) Applicant: WEINMANN EMERGENCY MEDICAL TECHNOLOGY GMBH + CO. KG, Hamburg (DE)

(72) Inventor: Tobias KALWA, Hamburg (DE)

(21) Appl. No.: 18/870,053

(22) PCT Filed: May 26, 2023
(86) PCT No.: PCT/DE2023/100395

§ 371 Date: Nov. 27, 2024

(30) Foreign Application Priority Data
Jun. 2, 2023 (DE) 10 2022 113 960.3

(51) Int. Cl. G16H 40/20; G16H 20/40; G16H 40/60; H04L 9/40

(52) U.S. Cl. CPC G16H 40/20; G16H 20/40; G16H 40/60; H04L 63/08

(57) ABSTRACT

A medical device, a medical system as well as a method for controlling a medical device and a method for determining the training needs of a user of medical devices, in which the user-specific usage data of at least one medical device is collected and determined by comparison with specifications whether the user has a need for training with at least one medical device or a certain operating mode. A user is automatically informed of any training needs after being identified on a medical device.

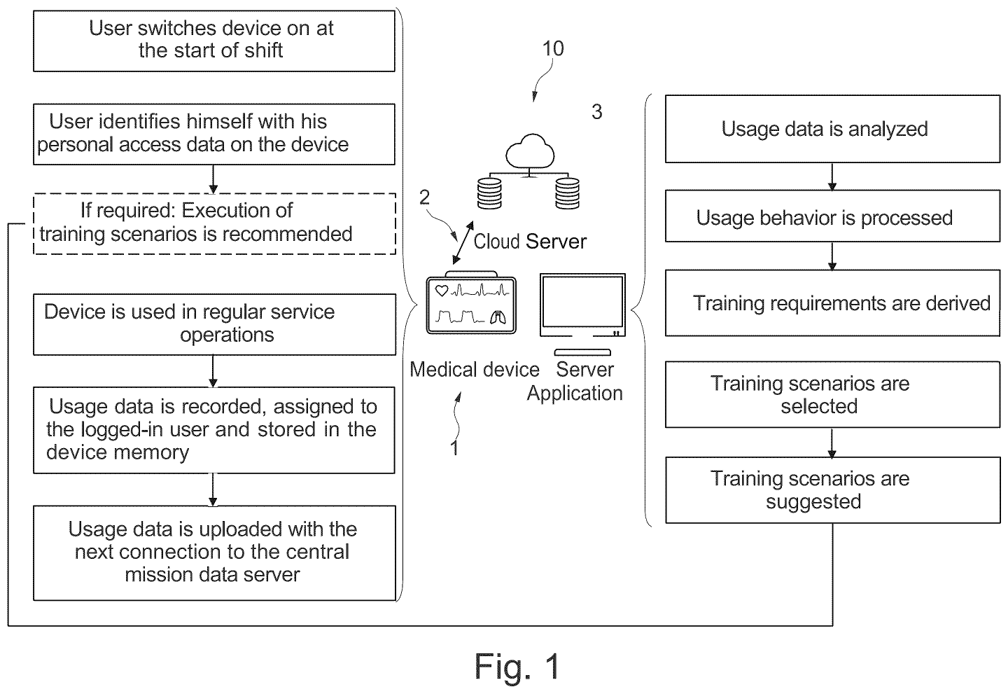

The invention relates to a medical device, a medical system comprising at least one medical device and a method for controlling a medical device.

Furthermore, the invention relates to a method for determining the training needs of a user of medical devices.

In the case of medical devices and especially in the case of medical devices for use in emergency medicine, such as ventilators or defibrillators, it is of considerable importance for the success of medical treatment that the skilled operation of the medical devices is mastered by the respective users.

Modern medical devices can be used to implement a variety of specific operating modes and functions, so that special operating modes or functions are usually rarely used in daily use.

In emergency medicine, the users of such medical devices are usually emergency responders such as paramedics, emergency paramedics, rescue assistants or emergency doctors. The skilled operation of the medical equipment can only be learned and trained or the corresponding knowledge be obtained through regular training and exercises and/or the use of the equipment in operations. It is therefore important to always determine the training needs of individual emergency responders and to train them on the respective medical equipment according to the identified needs.

According to the state of the art, for example, in rescue services, the person-specific data on the training status of the emergency responders with the individual medical devices that are used must be manually recorded, stored and processed in order to determine and monitor the respective training needs.

It is therefore an object of the invention to specify an improved medical device which solves at least some of the aforementioned problems.

According to the invention, this object is achieved by a medical device according to claim 1.

It is a further object of the invention to provide a medical system which solves at least some of the aforementioned problems.

This object is achieved by a medical system according to patent claim 9.

Futhermore, it is an object of the invention to provide a method for controlling a medical device which solves at least some of the aforementioned problems.

According to the invention, this object is achieved by a method for controlling a medical device according to claim 14.

It is also an object of the invention to provide a method for determining the training needs of a user of medical devices that solves at least some of the aforementioned problems.

According to the invention, this object is achieved by a method for determining the training needs of a user of medical devices according to claim 19.

Advantageous embodiments of the invention are claimed in the dependent claims.

The features of a medical device, a medical system and a method for controlling a medical device and a method for determining the training needs of a user of medical devices disclosed below are part of the invention both individually and in all practicable combinations.

A medical device according to the invention has at least one data acquisition module and one communication device.

In embodiments of the invention, the medical device, which is designed, for example, as a ventilator, a defibrillator, an automatic external defibrillator (AED), an ECG device or a monitor system, comprises a therapy and/or monitoring module.

A therapy module is designed for the medical treatment of a patient, for example by the controlled delivery of breathing air from a ventilator or through the delivery of electric shocks from a defibrillator.

A monitoring module is designed to measure, output and/or monitor at least one of a patient's vital parameters, such as pulse rate, blood oxygen saturation, an electrocardiogram, etc.

The data acquisition module is designed to acquire usage data of the medical device.

In embodiments of the invention, the data acquisition module is designed for the acquisition of usage data such as the usage time, the mode of operation used and/or operating parameters selected by the user.

The operating mode of medical devices designed as combined defibrillator-monitor systems can be, for example, pacing, cardioversion, defibrillation or capnography.

In preferred embodiments of the invention, the data acquisition module is designed to collect the usage data of the therapy and/or monitoring module.

In addition, the data acquisition module has an identification device for identifying a user using the medical device.

In embodiments of the invention, the identification device for the identification of the user is designed by capturing a login comprising at least a user-specific username and/or password, capturing the user's biometric data, and/or capturing a user-specific ID tag.

If the identification device is designed to identify the user by capturing a login, the identification device has input means or is designed to use input means of the medical device with which a user name and/or password can be entered. For example, the identification device and/or the medical device has a keyboard and/or buttons and/or switches and/or a touch display.

If the identification device is designed to identify the user by capturing biometric data of the user, the identification device has a biometric capture device or is trained to use a biometric capture device of the medical device. For example, the biometric capture device is designed as a fingerprint sensor and/or an iris scanner and/or a face scanner.

If the identification device is designed to identify the user by capturing a user-specific ID tag, the identification device has an ID tag reader or is trained to use a reader for ID tags of the medical device. For example, the ID tag reader is designed as a barcode or QR code scanner, as an NFC reader, or as a card reader.

In preferred embodiments of the invention, the identification device is designed for the comparison of the acquired identification data with the identification features of known users stored in a user database for identifying a user.

In embodiments of the invention, the data acquisition module is designed for acquiring usage data and linking the usage data to a user ID.

In preferred embodiments of the invention, the data acquisition module is also designed to link the usage data to a device ID.

The communication device is designed to receive and send data to an external evaluation and control unit, such as a server or web server with a corresponding application.

In embodiments of the invention, the communication device is designed for communication via Bluetooth, WLAN and/or a radio network such as a satellite radio network or a mobile phone network. In alternative embodiments, additional or only wired communication equipment is implemented.

In advantageous embodiments of the invention, the medical device has a training module that is designed to train users with regard to the use of the medical device.

For example, the training module is designed to simulate an operation in which the simulated patient is to be treated by a user using the medical device on the basis of simulated vital parameters of a patient simulated in this regard.

In preferred embodiments of the invention, the medical device is designed for the automated retrieval of user-specific data from an external database, for example a central database on a web server, so that after the identification of a user, the user can be automatically informed whether training with the respective medical device is necessary or will be required in the near future.

A medical system according to the invention has at least one medical device according to the invention and at least one evaluation and control unit which, in preferred embodiments of the invention, is located outside the medical technology device.

At least one medical device is connected to the evaluation and control unit for mutual data transmission.

In embodiments of the invention, the evaluation and control unit is realized by a web server with a corresponding application for evaluation and control.

In preferred embodiments with an evaluation and control unit located outside the medical device, aata transmission between at least one medical device and the evaluation and control unit is carried out via the Internet.

Particularly preferably, the medical system is designed for the encrypted transmission of data in order to ensure appropriate data security.

In embodiments of the invention, the evaluation and control unit is integrated into the medical device.

The evaluation and control unit is designed to receive and evaluate user-specific usage data from at least one medical device.

The user-specific usage data can be stored in a storage device and retrieved from the evaluation and control unit.

With the help of the evaluation and control unit, the user-specific usage data for one or more medical devices can be compared with predetermined values, and a training need can be determined for each user based on this comparison. User-specific usage data can be filtered using filter criteria.

For example, for a medical device, a filter criterion is set for a specific period of time, e.g. for 12 months prior to the current date, a usage time of the medical device of at least 10 hours is defined as a predetermined value. The value of the usage time stored in the user-specific usage data can then be filtered according to the filter criterion and is comparable to the predetermined value. If the considered usage time of the respective user is below the predetermined value of 10 hours, an existing training need is determined. If the usage time of the respective user is at least 10 hours, no need for training is determined.

The data on a user's training needs can be retrieved from at least one medical device from the evaluation and control unit and/or actively transmitted from the evaluation and control unit to the medical device or several medical devices.

According to the training needs of an identified user, the medical device is designed to output an existing training need of the user in order to prompt the user to train and/or start a training module.

In embodiments of the invention, the medical system comprises at least one individual ID tag per user.

A method for controlling a medical device according to the invention comprises at least the following steps:
1. Identification of the user of a medical device and determination of a user ID assigned to the user,
2. Collection of usage data during the use of the medical device by the user in regular operation and/or during training,
3. Linking the collected usage data with the user's recorded user ID,
4. Transfer of the linked data to an external evaluation and control unit,
5. Adding usage data to an existing record for the user or creating a new record for the user,
6. Evaluation of usage data for at least one user,
7. Comparison of at least one value of a user's usage data per medical device and/or operating mode of a medical device with predetermined values,
8. Deriving the need for training of the respective user with a medical device or a certain operating mode of a medical device,
9. Transmission of the user's training needs to the respective medical device or to several medical devices.

In preferred embodiments of the invention, the medical device offers the user appropriate training after identification on the medical device according to the user's training needs and/or generates an output to inform the user about an existing training need.

In a preferred embodiment of the method according to the invention, the medical device actively retrieves the user profile with the individual training needs stored therein after the user has been identified on the device. This procedure has the advantage that the user is not limited to a specific medical device with regard to information about a required training and the data on a user's training needs does not have to be transferred from the evaluation and control unit to all available medical devices as a precautionary measure.

In embodiments of the invention, the user profiles are initially created in a database accessible by the evaluation and control unit. The user ID is stored in the database so that the collected usage data can later be assigned to the user profile via the user ID.

For example, the user profiles are created and managed by the management of the rescue service on a web platform.

In embodiments of the invention, at least one medical device regularly connects to this web platform and synchronizes the usage data of the respective user.

The identification of the user on the medical device is possible, for example, in the following variants. According to variant 1, the user logs on to the medical device by entering the personal ID assigned to him (e.g. username and/or password) at the beginning of the shift. According to variant 2, the user logs on to the medical device at the beginning of the shift with the help of authentication using an NFC tag.

For example, the following variants are conceivable for the user to log out of the medical device. According to variant 1, the user manually logs out of the medical device at the end of the shift, for example by pressing an analogue or digital log-out button. According to variant 2, the user logs out of the medical device at the end of the shift by reading his individual NFC tag. According to variant 3, the user is automatically logged out at a fixed time. The respective time can be determined, for example, by the management of the rescue service.

When the usage data is collected, the data is stored locally on the medical device, at least temporarily.

As soon as the user-specific usage data has been transferred to the evaluation and control unit, it is stored there in a database permanently or at least for a period of at least 12 months.

The user-specific usage data stored locally on the medical device are deleted in embodiments of the invention as soon as they have been transferred to the evaluation and control unit.

In embodiments of the invention, the user-specific usage data are exchanged every time the medical device connects to the evaluation and control unit, even if the reason for establishing the connection is different (e.g. uploading function control results, telemetry application, etc.).

In preferred embodiments of the invention, the medical device automatically checks whether the identified user has identified a need for training on the medical device, after the identification of the user.

The determination of a user's training need with a specific medical device or a specific function of a medical device is carried out in embodiments of the method according to the invention by comparing user-specific usage data with predetermined values for certain usage data, whereby a training need is determined if a usage criterion is not met. In particular, this may be the case if the respective value of the user-specific usage data is below a predetermined minimum value.

In preferred embodiments of the method of the invention, the usage data of at least one user stored in the database can be filtered by means of at least one filter criterion.

Such a filter criterion can be, for example, a certain period of time, a certain medical device, or a certain functionality of a medical device.

In embodiments, the evaluation and control unit is used to compare detailed usage data of at least one user with at least one target specification (e.g. the course of a treatment with a predetermined sequence of treatment steps and/or predefined setting values). Based on this comparison, the quality of the treatment(s) performed by the user is evaluated and a quality index is assigned.

In advantageous embodiments of the invention, a training need of the user is determined from the quality assessment by comparing the respective quality index with a specified minimum value for the quality index, if the quality index is below the specified minimum value.

In preferred embodiments of the method of controlling a medical device according to the invention, a medical device or a medical system according to the invention is used.

A method according to the invention for determining the training needs of a user of medical devices comprises at least the following method steps
1. Identification of the user and determination of the user ID,
2. Collection of usage data during the use of the medical device in regular operation and/or for training,
3. Linking the collected usage data to the user ID,
4. Transfer of the linked data to an external evaluation and control unit,
5. Adding usage data to an existing record for the user or creating a new record for the user,
6. Evaluation of usage data for at least one user
7. Comparison of usage data per medical device and/or operating mode of a medical device with predetermined values
8. Deriving the need for training of the respective user with a medical device or a certain operating mode of a medical device
9. Output of the determined training needs for each user and device to one or more responsible persons.

The responsible persons according to step 9 are in particular persons working in the organization and/or management of rescue services who inform respective users about a training need/organize training for and/or with the respective users.

In embodiments of the invention, the user-specific usage data is transferred to a web portal. There, the data for each user is graphically processed in such a way that, for example, the rescue service management can see what the device usage level of the respective user is. This allows the rescue service management to deduce where there is a need for training.

Usage data within the meaning of this invention are in particular those data which, individually or in combination, can provide information as to whether and how a medical device has been used by a user.

For example, such usage data is selected individually or in any combination from the following data:
The device runtime
The number of missions with the medical device
The runtime of the different operating modes
   for a defibrillator monitor: e.g. AED mode, monitor mode, manual mode, pacemaker mode)
   for an AED: e.g. AED mode, manual mode
   for a ventilator: e.g. ventilation mode (IPPV, CPAP, SIMV, PCV, bi-level, etc.), NIV mode, CPR mode, RSI mode
The number of defibrillations performed
The number and duration of CPR feedback sensor uses
The number of 12-lead diagnostic ECGs
The number of functional checks carried out
The number of live data connections
The number and duration of menu usages
The number and duration of replay views
The number and type of printouts
The number of blood pressure measurements taken
The number of data transfers to an external documentation system
The number of data transfers to a central mission data server
The number of entries into ventilation via the emergency mode
The number of admissions to ventilation via patient data such as patient size and gender
The duration of the display of curves (e.g. ECG, flow, CO2, pressure, etc.)

In particular in such embodiments of the invention, the usage data is linked to an external evaluation and control unit for use with a number of different medical devices in addition to the user ID of the respective user in addition to a device ID. This device ID is defined either individually for each individual medical device or individually for groups of the same medical device (e.g. five ventilators of the same type). This makes it possible to distinguish between different medical devices in the database in which the user-specific usage data is stored.

In embodiments of the invention, one or more different training sessions are available on at least one medical device, on the basis of which the user can be trained if necessary. Each training session is linked to certain attributes that can be used to increase the level of training differentiated according to the specific types of equipment use. If, during the evaluation of the use of the device, it is determined with the help of the evaluation and control unit that there is a deficit (e.g. no defibrillation in the last year, no pacemaker therapy in the last year, only 2x start of ventilation via height and gender, etc.), the user is suggested to carry out the corresponding training session by means of a corresponding output on the medical device. In embodiments of the invention, the degree of device usage from which a training session is suggested to the user can be set by adjusting the specified values and/or filter criteria by the rescue service management.

The figure explained below illustrates an embodiment of a medical system according to the invention (10) and the sequence of an embodiment of a method for controlling a medical device (1) according to the invention.

The medical device (1) is designed as a defibrillator monitor and is connected to an evaluation and control unit (3) designed as a cloud server via a communication interface (2), in this case designed as a connection via the Internet. A server application is installed and started on the cloud server, which implements the necessary process steps on the evaluation and control unit (3).

According to the procedure described above, a user first switches on the medical device (1). The user then identifies himself at the medical device (1). After identification, the medical device (1) automatically checks whether the identified user needs training on or with the medical device (1) by querying the user-specific data on the training need from the evaluation and control unit (3). If there is a need for training with the medical device, the user is recommended training by means of an automatic output. The medical device (1) is then used by the user in regular service or for training. The usage data is recorded, assigned to the identified user and stored in the memory of the medical device (1). The next time the user is connected to the evaluation and control unit (3), the usage data linked to the user ID and, if applicable, also to the device ID are transmitted to the evaluation and control unit (3) and stored. The usage data is then evaluated in a further procedural step, the user behavior is processed and any training needs are derived. Based on the derived training needs for the respective user, suitable training scenarios are automatically selected and suggested to the user on a suitable medical device after identification.

1-20. (canceled)

21. A medical device, comprising: at least one data acquisition module for recording usage data of the medical device; and a communication device for sending data to or receiving data from an external evaluation and control unit, wherein the data acquisition module includes an identification device for identifying a user using the medical device.

22. The medical device according to claim 21, further comprising at least one therapy and/or monitoring module.

23. The medical device according to claim 22, wherein the data acquisition module is configured to collect the usage data of the therapy and/or monitoring module.

24. The medical device according to claim 21, wherein the identification device is configured to identify the user by capturing a login comprising at least a user-specific username and/or password, capturing the user's biometric data, and/or capturing a user-specific ID tag.

25. The medical device according to claim 21, wherein the data acquisition module is configured to collect usage data and to link the usage data to a user ID.

26. The medical device according to claim 25, wherein the data acquisition module is further configured to link the usage data with a device ID.

27. The medical device according to claim 21, further comprising a training module configured to train users with regard to use of the medical device.

28. The medical device according to claim 21, wherein the medical device is configured to automatically retrieve user-specific data from an external database.

29. A medical system, comprising: at least one medical device according to claim 21; and at least one evaluation and control unit, wherein the at least one medical device is connected by a communication interface to the evaluation and control unit for mutual data transmission, wherein the evaluation and control unit is configured to receive and evaluate user-specific usage data from the at least one medical device, wherein the user-specific usage data is storable in a storage device by and from the evaluation and control unit, wherein the evaluation and control unit compares the user-specific usage data for the at least one medical device with predetermined values so that a training need is determinable for a user based on the comparison, wherein the data of training needs of the user is retrievable by the at least one medical device from the evaluation and control unit and/or is actively transmitted from the evaluation and control unit to the at least one medical device, and wherein the at least one medical device is configured to output any existing training needs of the user to prompt the user to train and/or start a training module, according to the training needs of the user.

30. The medical device according to claim 29, wherein the evaluation and control unit is located outside of the at least one medical device.

31. The medical device according to claim 30, wherein the evaluation and control unit is a web server with a corresponding application for evaluation and control.

32. The medical device according to claim 29, wherein the evaluation and control unit is configured to filter the user-specific usage data based on at least a filter criterion.

33. The medical device according to claim 29, further comprising at least one individual ID tag per user.

34. A method for controlling a medical device, comprising the steps of:
a. Identifying a user of a medical device and determining a user ID assigned to the user;
b. Collecting usage data during use of the medical device by the user in regular operation and/or during training;
c. linking the collected usage data with the user ID collected;
d. Transferring the linked data to an external evaluation and control unit;
e. Adding usage data to an existing record for the user or creating a new record for the user;
f. Evaluating usage data for at least one user;
g. Comparing at least one value of a user's usage data per medical device and/or operating mode of a medical device with predetermined values;
h. Deriving a need for training of a respective user with a medical device or a specific operating mode of a medical device; and transmitting
i. Transmitting the user's training needs to the respective medical device or to several medical devices.

35. The method for controlling a medical device according to claim 34, including offering, using the medical device, the user appropriate training in accordance with the user's training needs and/or generates an output to inform the user about an existing training need after the user's identification on the medical device.

36. The method for controlling a medical device according to claim 34, including actively retrieving, using the medical device, the user profile with the individual training needs stored therein after the identification of the user on the medical device.

37. The method for controlling a medical device according to claim 34, including regularly connecting the medical device to the evaluation and control unit and synchronizing the usage data of the respective user.

38. The method for controlling a medical device according to claim 34, including using a medical system comprising: at least one medical device comprising at least one data acquisition module for recording usage data of the medical device, and a communication device for sending data to or receiving data from an external evaluation and control unit, wherein the data acquisition module includes an identification device for identifying a user using the medical device; and at least one evaluation and control unit, wherein the at least one medical device is connected by a communication interface to the evaluation and control unit for mutual data transmission, wherein the evaluation and control unit is configured to receive and evaluate user-specific usage data from the at least one medical device, wherein the user-specific usage data is storable in a storage device by and from the evaluation and control unit, wherein the evaluation and control unit compares the user-specific usage data for the at least one medical device with predetermined values so that a training need is determinable for a user based on the comparison, wherein the data of training needs of the user is retrievable by the at least one medical device from the evaluation and control unit and/or is actively transmitted from the evaluation and control unit to the at least one medical device, and wherein the at least one medical device is configured to output any existing training needs of the user to prompt the user to train and/or start a training module, according to the training needs of the user.

39. The method for determining training needs of a user of a medical device, comprising the steps of:
a. Identifying the user and determining a user ID;
b. Collecting usage data during use of the medical device in regular operation and/or for training;
c. linking the collected usage data to the user ID;
d. Transferring the linked data to an external evaluation and control unit;
e. Adding usage data to an existing record for the user or creating a new record for the user;
f. Evaluating usage data for the user;
g. Comparing usage data per medical device and/or operating mode of the medical device with predetermined values;
h. Deriving a need for training of the user with the medical device or a specific operating mode of the medical device; and
i. Outputting the derived training needs of the user for the medical device.

40. The method for determining the training needs of a user of a medical device according to claim 39, including using a medical device comprising: at least one data acquisition module for recording usage data of the medical device; and a communication device for sending data to or receiving data from an external evaluation and control unit, wherein the data acquisition module includes an identification device for identifying a user using the medical device.
